(19) United States
(12) Patent Application Publication
(10) Pub. No.: US 2012/0231097 A1
(43) Pub. Date: Sep. 13, 2012

(54) MEDICINAL COMPOSITION AND USAGE

(75) Inventors: James Zhou, Beijing (CN); Dong Chen, Beijing (CN)

(73) Assignee: James Zhou, Beijing (CN)

(21) Appl. No.: 13/512,446

(22) PCT Filed: Nov. 25, 2010
(86) PCT No.: PCT/CN2010/079105

§ 371 Date: May 29, 2012

(30) Foreign Application Priority Data
Nov. 26, 2009 (CN) 200910237946.9

(51) Int. Cl. A61K 36/539; A61P 1/04; A61P 35/00; A61K 31/7034

(52) U.S. Cl. 424/745; 514/35

(57) ABSTRACT

This invention provides a traditional Chinese medicine composition consisting of the extract of no more than five crude drugs, wherein crude drugs include licorice and also include Radix Scutellariae or Rhizoma Polygoni Cuspidati. In addition, this invention also provides the preparations, treatment methods, treatment applications and preparation methods of the above traditional Chinese medicine compositions.

### TECHNICAL FIELD

This invention belongs to traditional Chinese medicine technical field; it involves a kind of traditional Chinese medicine composition, which has evident therapeutic effect for oral ulcer, and can also improve the effectiveness of cancer chemotherapy and lower the toxic and side effects of chemical medicine.

### BACKGROUND TECHNOLOGY

Backed by thousands of years of development history and profound theoretical background, traditional Chinese medicine advocates holistic treatment, and pair-up combination in drug usage, namely differentiating drug property based on “four properties and five tastes” of drugs; meanwhile it also makes reference to the principle of attributive channel for illnesses to differentiate the attributive channels of traditional Chinese medicine. Traditional Chinese pharmacist usually lists out one or several effective drugs based on symptoms, and match them up based on these drugs' attributive channel, five tastes and four properties in subordinate sequence, namely the commonly said ‘monarch, minister, assistant and messenger’, so as to develop Chinese herbal medicine prescription with miraculous efficacy. Nevertheless, over the past several thousands of years, although traditional Chinese medicine has proved its therapeutic effects, its quality varies depending on different individuals and processing methods, so that drug quality and its efficacy are hardly consistent, in particular, traditional Chinese medicine formula will usually include over a dozen or even several dozen (i.e., variety) crude drugs, which complicates the factors affecting processing quality. For instance, China Patent Application No. 02100168 and No. 00113577 respectively published traditional Chinese medicine or healthcare products made from Radix Scutellariae, Rhizoma Polygoni Cuspidati, licorice, Radix puerariae, Semen Celosiae and other crude drugs totaling over a dozen crude drugs, which are used to reduce blood lipid and weight.

Oral ulcer is a common oral disease, its occurrence rate is 10% of the total population. Its clinical symptoms are repeated occurrences of isolated, round or oval shaped superficial ulcer in oral mucous membrane, and local burning pain. At present, drugs used to treat oral ulcer are mostly exterior-syndrome relieving western medicine, which alleviate pain, but cannot cure the disease, there are few effective traditional Chinese medicine preparations.

Excluding operation, the treatment methods for tumors are still radiation therapy and chemotherapy. In most cases, a combined way to integrate operation, radiation and chemical therapy will be used. The toxic and side effects of radiation and chemical therapy will bring, so called side effects, great harm to human body, in particular it will bring serious damage to human body's immunity system and hematopoietic cells. Meanwhile, because tumor cells will develop drug resistance, it is difficult to thoroughly remove all tumor cells from human body by using chemotherapy means. Consequently, after radiation and chemical therapy treatment, the recurrence rate is very high. After relapse it is even more difficult to use radiation and chemical therapy means to bring about cure, which results in the situation that after relapse the condition of most tumor patients will deteriorate to uncontrollable degree and even endanger life. Treating tumor with Chinese herbs is expected to avoid the disadvantages of toxicity in radiation and chemotherapy and tumor cell drug resistance, this is because Chinese herbs contain a variety of ingredients, and highlight combination to improve synergy therapy effect and overall treatment effects. While killing tumor cells, it can protect human body's hematopoietic cell and immunity system. On the other hand, it will activate human body's certain immunity functions to fulfill self control or remove tumor cells. Some Chinese proprietary medicines whose effects are recognized in reducing toxic and side effects of radiation and chemical therapy are already in use. However, the difficulty of using traditional Chinese herb to treat tumor is: Uncertain therapeutic effect, and the lack of effective method to produce Chinese proprietary medicine with stable quality and implement quality control. Some effective chemical monomers extracted from Chinese herb drugs or plants can overcome the difficulty of Chinese herb medicine for treating tumor as anti-tumor drug. However, according to such “westernization” of traditional Chinese medicine, when effective chemical monomer is made into medicine, due to lack of synergy effects among different active ingredients, they will on the contrary decrease the therapeutic effect. Through long time study and practice on traditional Chinese medicine in traditional theory, modern pharmacy, plant chemistry fields, this inventor has unexpectedly obtained new traditional Chinese medicine composition, which only needs 2 to 5 kinds of specific crude drugs to quickly and effectively cure oral ulcer, meanwhile it can treat (especially assist the chemotherapy treatment of) cancer, so as to improve chemotherapy effects, and alleviate chemotherapy's side effects. After making exploration, this inventor has also obtained the preparation method of this traditional Chinese medicine composition; since it greatly reduces the variety (kind) of crude drugs, it is easier to control the production quality of the traditional Chinese medicine composition in this invention, so that the produced traditional Chinese medicine has more stable therapeutic effect.

### INVENTION DETAILS

This invention aims to provide a kind of traditional Chinese medicine composition, which has evident therapeutic effects for oral ulcer, and can also improve the effect of cancer chemotherapy and reduce toxic and side effects of chemical medicine. Furthermore, this invention also aims to provide application for such traditional Chinese medicine composition.

Specifically, in the first aspect, this invention provides a traditional Chinese medicine composition consisting of extracts of no more than five crude drugs, wherein the crude drugs include licorice and also Radix Scutellariae or Polygonum cuspidatum, the preferred crude drugs include Radix Scutellariae, Polygonum cuspidatum and licorice, the most preferred crude drugs include Radix Scutellariae, Polygonum cuspidatum, licorice, Radix Puerariae and Cassia seed. “Crude drug” possesses the conventional meaning understood by technicians in the field of traditional Chinese medicine, namely natural unprocessed or briefly processed (e.g. drying, slicing, crushing etc) plant-derived Chinese herb medicine. Crude drugs are often incorporated into traditional Chinese medicine composition in the form of crude drug extracts after passing extraction to obtain concentrated active ingredients. The variety of crude drugs needed by the traditional Chinese medicine composition in this invention does not exceed five kinds. The less the variety of crude drugs, the more will it be possible to reduce the complexity degree of production process in the preparation of traditional Chinese medicine composition, so as to facilitate the control of production quality, and improve the quality of the composition. In the embodiments of this invention, the traditional Chinese medicine composition consists of Radix Scutellariae extract, lygonum cuspidatum extract and licorice extract; or Polygonum cuspidatum extract and licorice extract; the preferred crude drugs consist of Radix Scutellariae extract, Polygonum cuspidatum extract and licorice extract, the most preferred crude drugs consist of Radix Scutellariae extract, Polygonum cuspidatum extract, licorice extract, Radix Puerariae extract and Cassia seed extract. Through experiments, the inventor has discovered that, as the variety of crude drugs needed by the preparation of traditional Chinese medicine composition increases, the corresponding therapeutic results will be further improved, which reflects the synergy effect between different ingredients.

In the traditional Chinese medicine composition in this invention, the preferred weight ratio of crude drugs is: licorice 0.5-2.5 portions, Radix Scutellariae 1-5 portions, Rhizoma Polygoni Cuspidati 0.5-5 portions, Radix puerariae 0.2-2.5 portions, and/or Semen Celosiae 0.3-2.5 portions. Wherein, “and/or” refers to the case when the traditional Chinese medicine composition needs corresponding crude drugs varieties for preparation, its weight ratio is taken into consideration; when the traditional Chinese medicine composition does not need corresponding crude drugs varieties for preparation, then its weight ratio is not taken into consideration. For instance, when the traditional Chinese medicine composition consists of Radix Scutellariae extract and licorice extract, the preferred weight ratio of Radix Scutellariae and licorice is 1-5: 0.5-2.5; when the traditional Chinese medicine composition consists of Rhizoma Polygoni Cuspidati extract and licorice extract, the preferred weight ratio of Rhizoma Polygoni Cuspidati and licorice is 0.5-5: 0.5-2.5; when the traditional Chinese medicine composition consists of Radix Scutellariae extract, Rhizoma Polygoni Cuspidati extract and licorice extract, the preferred weight ratio of Radix Scutellariae, Rhizoma Polygoni Cuspidati and licorice is 1-5: 0.5-5: 0.5-2.5; when the traditional Chinese medicine composition consists of Radix Scutellariae extract, Rhizoma Polygoni Cuspidati extract, licorice extract, Radix puerariae extract and Semen Celosiae extract, the preferred weight ratio of Radix Scutellariae, Rhizoma Polygoni Cuspidati, licorice, Radix puerariae and Semen Celosiae is 1-5: 0.5-5: 0.5-2.5: 0.2-2.5: 0.3-2.5. The most preferred weight ratio is shown in the Formula listed in the embodiments of this invention.

Radix Scutellariae, also known as Huangqin in Chinese, is a kind of Chinese herb available on market, its extract includes baicalin, baicalein, wogonoside, wogonin, skullcapflavone I and II, oroxy-lin A and Campesterol etc. In the traditional Chinese medicine composition of this invention, Radix Scutellariae extract can be obtained from Radix Scutellariae through conventional methods, the extraction methods include ultrasonic extraction method and acid subsidence (sedimentation) method, the preferred way to obtain Radix Scutellariae extract is extraction through acid precipitation method. Acid precipitation methods include water extraction acid precipitation method, alkaline extraction acid precipitation method and ethanol extraction acid precipitation method, namely using water, alkaline solution or ethanol solution to extract Radix Scutellariae, then add acid (e.g. hydrochloric acid) for sedimentation. In this invention, it is possible to determine the quality of Radix Scutellariae extract based on baicalin's content. Preferably the extracted baicalin content in Radix Scutellariae extract should exceed 10% (weight), the more preferred percentage is over 50% (weight).

Rhizoma Polygoni Cuspidati, also known as Hu Zhang in Chinese, is a kind of Chinese herb available on market, its extract includes emodin, rheic acid, chrysophanol, emodin-6-methyl ether, emodin-8-oxo-D Glucoside (i.e., polydatin), resveratrol, Piceid, 7-Hydroxy-4-methoxymethylcoumarin, Torachryson-8-oxo-D-Glucoside, reenoutrin, flavonol, isoquercitrin and glucofuranose etc. In the traditional Chinese medicine composition of this invention, Rhizoma Polygoni Cuspidati extract can be extracted from Rhizoma Polygoni Cuspidati through conventional ways, the extraction methods include ethanol extraction method and water extraction method, namely using ethanol or water to extract Rhizoma Polygoni Cuspidati, then remove solution. The preferred way to obtain Rhizoma Polygoni Cuspidati extract is through ethanol extraction method; the most preferred is extraction by using 95% (volume) ethanol. In this invention, it is possible to use polydatin content to determine the quality of Rhizoma Polygoni Cuspidati extract. Preferably the polydatin content in Rhizoma Polygoni Cuspidati extract after extraction should exceed 1% (weight), the preferred concentration exceeds 2% (weight).

Licorice (Radix Glycyrrhizae), also known as Gan Cao in Chinese, is a kind of Chinese herb which is available on market, its extract includes Glycyrrhizic acid, licoflavone, liquiritigenin, liquiritin, glabridin and chalcone etc. Licorice varieties include Glycyrrhiza uralensis, Glycyrrhiza Glabra and Glycyrrhiza inflata Batalin etc. In the traditional Chinese medicine composition of this invention, licorice extract can be extracted from licorice through conventional ways, the extraction methods include ethanol extraction method and water extraction method, namely use ethanol or water to extract licorice, then remove the solution. The preferred way to obtain licorice extract is through ethanol extraction method, the most preferred is to use 95% (volume) ethanol for extraction. In this invention, it is possible to determine the quality of licorice extract based on the content of licoflavone. The preferred case is after extraction, the licoflavone content in licorice extract exceeds 4% (weight).

Radix puerariae, also known as Ge Gen in Chinese, is a kind of Chinese herb available on market, its extract includes Puerarin, isoflavoues Aglycone, Daidzin etc. In the traditional Chinese medicine composition of this invention, Radix puerariae extract can be extracted from Radix puerariae through conventional ways, the extraction methods include ethanol extraction method and water extraction method, namely using ethanol or water to extract Radix puerariae, then remove solution. The preferred way to obtain Radix puerariae extract is through ethanol extraction method, the most preferred way is extraction with 60% (volume) ethanol.

Semen Celosiae/Catsia tora Linn, also known as Jue Ming Zi in Chinese, is a kind of Chinese herb available on market, its extract includes emodin, chrysophanol, physcion, obtusin, and obtusifolin etc. In the traditional Chinese medicine composition of this invention, Semen Celosiae extract can be extracted from Semen Celosiae through conventional method, the extraction methods include ethanol extraction method and water extraction method, namely use ethanol or water to extract Semen Celosiae, then remove solution. The preferred way to obtain Semen Celosiae extract is through ethanol extraction method, the most preferred way is to use 60% (volume) ethanol for extraction.

On the other hand, this invention provides methods to prepare the composition described by this invention in the first aspect, which include:

(1) Extract Radix Scutellariae crude drugs to obtain Radix Scutellariae extract, the preferred way is to extract through acid precipitation method;

(2) Extract Rhizoma Polygoni Cuspidati crude drugs to obtain Rhizoma Polygoni Cuspidati extract, the preferred way is to extract through ethanol extraction method;

(3) Extract licorice crude drugs to obtain licorice extract, the preferred way is to extract through ethanol extraction method;

(4) Extract Radix puerariae crude drugs to obtain Radix puerariae extract, the preferred way is to extract through ethanol extraction method or water extraction method;

(5) Extract Semen Celosiae crude drugs to obtain Semen Celosiae extract, the preferred way is to extract through ethanol extraction method or water extraction method;

(6) Mix up the extracts obtained from Step (3) and (1) and/or (2) and/or (4) and/or (5) and/or (6).

Step (6) does not need mixed extracts, before mixing there is no need to prepare. Wherein, the preferred case in Step (6) is to mix up the extracts obtained through Step (1)-(6).

Acid precipitation method, ethanol extraction method and water extraction method are all methods known to technicians in this field, due to different extracted solution, in the obtained extract the effective ingredients might differ. This invention prefers acid precipitation method for Radix Scutellariae crude drugs; prefers ethanol extraction method for Rhizoma Polygoni Cuspidati crude drugs; prefers ethanol extraction method for licorice crude drugs; prefers ethanol extraction method or water extraction method for Radix puerariae crude drugs, especially ethanol extraction method; and/or, prefers ethanol extraction method or water extraction method for Semen Celosiae crude drugs, especially ethanol extraction method. The embodiments of this invention provide exemplary extraction methods, technicians in this field can obtain other real extraction methods based on these descriptions under the framework of acid precipitation method, ethanol extraction method and water extraction method.

Secondly, this invention provides traditional Chinese medicine preparations, which include the composition described by the first aspect of this invention and pharmaceutically acceptable adjuvant. Include pharmaceutically acceptable vehicle, excipient, diluent, etc; they are compatible with active ingredients. Use pharmaceutically acceptable adjuvant. Preparation of drug composition is known to common technicians in this field. The traditional Chinese medicine preparation in this invention includes the composition described by the first aspect of this invention as active ingredients, it combines such composition and pharmaceutically acceptable adjuvant. (e.g. vehicle, excipient, diluent etc familiar to common technicians in this field), to make into various preparations, the preferred form is solid preparation and liquid preparation. The traditional Chinese medicine preparation in this invention can adopt the form of unit dosage form, such as tablet, pill, capsule (including sustained release or delayed release forms), powder, mixed suspension agent, particle, tincture, syrup, emulsion, suspension solution, spray preparations, so as to adapt to various intake methods, for instance oral intake, non-intestine injection, membrane, muscle, intravenous, subcutaneous, intraocular, intracutaneous or via skin intake methods.

The preferred preparation of traditional Chinese medicine dose in this invention is oral intake dose. Accordingly, vehicle, excipient, diluent are medically accepted and compatible with active ingredients in oral intake. The preferred traditional Chinese medicine preparations are selected from spray, tablet (including buccal tablet), capsule, and oral intake liquid. In the actual embodiments of this invention, the composition described in the first aspect of this invention can dissolve in water glycerine solution to be made into liquid preparation.

Thirdly, This invention provides the usage of the composition described in the first aspect of this invention in drug preparation, the said drug is used to treat oral ulcer, or used for treating tumor, such as those used for treating colon cancer.

Fourthly, this invention provides the methods for treating oral ulcer or treating tumor (e.g., colon cancer), it includes the composition described in the first aspect of this invention which has efficacy in treating patients.

In this article, “treatment” includes unaided treatment and adjuvant treatment. In this invention, the composition described in the first aspect of this invention can be used independently as effective ingredient for treating oral ulcer or tumor (e.g. colon cancer), and can also be used in conjunction with other drugs (e.g. chemotherapy medicine, preferably 5-fluorouracil) for treating tumor (e.g. colon cancer). In the embodiments of this invention, the composition described in the first aspect of this invention and 5-fluorouracil are used together to improve 5-fluorouracil's therapeutic effects, it can also reduce the side effects brought by 5-fluorouracil toxicity.

The intake dosage and form are generally determined by the physician based on the patient's actual conditions (such as age, weight, sex, disease contracting time, body condition etc). Generally speaking, based on the composition described in the first aspect of this invention, the intake dosage is 0.01-10 g/kg patient weight, the preferred range is 0.05-1 g/kg, the more preferred is 0.1-0.5 g/kg. The intake form is determined based on the dosage of various drug preparations and its bio-availability, the most suitable intake form is oral intake.

The beneficial effects of this invention lie in the fact that, the traditional Chinese medicine composition in this invention can treat oral ulcer and tumor, in particular it can improve the therapeutic effect of chemotherapy drugs and lower the toxicity of chemotherapy drugs; in this invention, there is relatively few crude drugs used by traditional Chinese medicine composition, therefore it can facilitate production, and also facilitate quality control in the production, so as to stabilize the quality of the traditional Chinese medicine composition being produced.

To help with comprehension, the following text will describe this invention through actual embodiments. It is worth mentioning that, these descriptions are only exemplary description, and are not intended as limitation on the scope of this invention. Furthermore, this invention has quoted published literature (such as specific patents etc), these literatures also aim to describe this invention more clearly, all their textual contents are incorporated into this invention for reference, in much the same way that their full texts have been reiterated in the specifications of this invention.

### EMBODIMENTS

The following text uses real examples to further illustrate this invention's preparation formula and its preparation method, but it is not intended in any way to bring restriction on the scope of this invention.

### Embodiment 1

### Formula A (Five Crude Drugs Formula) and its Preparation Process

Crude drug Formula A: Radix Scutellariae 5 g, Rhizoma Polygoni Cuspidati 5 g, licorice 2.5 g, Radix puerariae 2.5 g, and Semen Celosiae 2.5 g.

Preparation method:

Take dried drug material Radix Scutellariae 500 g, after washing and crushing, add 2500 ml 70% (volume) ethanol to boil for 1 hour under 80-85° C., then filter it, keep the filter liquor; add 2500 ml 70% (volume) ethanol to filter residue and boil for 1 hour under 80-85° C., then filter it, kept the filter liquor. Combine the two filter liquors and heat up to concentrate till 500 ml , lower the temperature to 40-45° C., add hydrochloric acid to adjust the pH value of the filtered solution to 2, place it aside for 8 hours under room temperature, then filter it, keep the sediment. Subside the sediment with water till neutral, bake it in oven under 50-55° C. till dry, crushed into fine powder, then obtain Radix Scutellariae extract 32 g, Upon examination, its baicalin content is 643 mg/g.

Take dried drug material Rhizoma Polygoni Cuspidati 500 g, after washing and crushing, add 3000 ml water to soak at 30-35° C. for 8 hours, then filter it, keep the sediment. After sedimentation and water rinsing, bake it in oven under 50-55° C. till dry, then add 5000 ml 95% (volume) ethanol to reflux for 1 hour under 85° C., filter and keep the filter liquor. Steam off the ethanol (recyclable) in the filter solution, bake the solid in oven under 50-55° C. till dry, crush it into fine powder, to obtain Rhizoma Polygoni Cuspidati extract 47 g, after test its polydatin content is 22 mg/g.

Take dried drug material licorice 250 g, after washing and crushing, add 1500 ml water to soak at 30-35° C. for 8 hours, then filter it, keep the sediment. After sedimentation and rinsing, bake it in oven under 50-55° C. till dry, then add 5000 ml 95% (volume) ethanol to reflux for 1 hour under 85° C., filter and kept the filter liquor. Steam away the ethanol (recyclable) in the filter liquor, bake the solid in oven under 50-55° C. till dry, crush it into fine powder, to obtain licorice extract 13 g, after test its licoflavone content is 58 mg/g.

Take 250 g dried drug material Radix puerariae, after washing and crushing, add 4000 ml 60% (volume) ethanol to reflux for 3 hours under 85° C., filter and kept the filter liquor. Steam off the ethanol (recyclable) in the filter liquor, bake the solid in oven under 50-55° C. till dry, crush it into fine powder, to obtain Radix puerariae extract 37 g.

Take dried medicine drug material Semen Celosiae 250 g, after washing and crushing, add 4000 ml 60% (volume) ethanol to reflux for 3 hours under 85° C., filter and kept the filter liquor. Steam off the ethanol (recyclable) in filter liquor, bake the solid in oven under 50-55° C. till dry, crush it into fine powder, to obtain Semen Celosiae extract 18 g.

Then, evenly mix up the above prepared Radix Scutellariae extract, Rhizoma Polygoni Cuspidati extract, licorice extract, Radix puerariae extract and Semen Celosiae extract, to obtain traditional Chinese medicine composition in Formula A.

### Embodiment 2

### Formula B (Three Ingredients Crude Drugs Formula) and its Preparation Process

Crude drug Formula B: Radix Scutellariae 5 g, Rhizoma Polygoni Cuspidati 5 g, and licorice 2.5 g.

Preparation method: Based on the method described in Embodiment 1, respectively prepare Radix Scutellariae extract, Rhizoma Polygoni Cuspidati extract and licorice extract, then mix to obtain traditional Chinese medicine composition in Formula B.

### Embodiment 3

### Formula C (Decomposed Recipe Crude Drugs Formula 1) and its Preparation Process

Crude drug Formula C: Radix Scutellariae 5 g and licorice 1.25 g.

Preparation method: Based on the method described in Embodiment 1 respectively prepare Radix Scutellariae extract and licorice extract, then mix to obtain traditional Chinese medicine composition in Formula C.

### Embodiment 4

### Formula D (Decomposed Recipe Crude Drugs Formula 2) and its Preparation Process

Crude drug Formula D: 5 g of Rhizoma Polygoni Cuspidati and 1.25 g of licorice.

Preparation method: Based on the method described in Embodiment 1 respectively prepare Rhizoma

TABLE 1
Comparison of symptoms after drug in-take

| | | | 12 hours after | | 3 days after | |
|---|---|---|---|---|---|---|
| | | | treatment | | treatment | |
| | | | Ulcer | Pain | Ulcer | Pain |
| Patient | Sex/ | Drug | healing | alleviation | healing | alleviation |
| No. | Age | intake | % | % | % | % |
| 09-1 | F/25 | A | 0 | 80 | 75 | 90 |
| 09-2 | F/18 | A | 0 | 70 | 65 | 90 |
| 09-3 | F/23 | A | 0 | 75 | 75 | 85 |
| 09-4 | M/36 | A | 0 | 70 | 70 | 95 |
| 09-5 | M/45 | A | 0 | 80 | 80 | 85 |
| Average | | A | 0 | 75 | 73 | 89 |
| 09-6 | M//19 | B | 0 | 50 | 65 | 70 |
| 10-7 | F/31 | B | 0 | 50 | 50 | 65 |
| 10-8 | F/28 | B | 0 | 50 | 70 | 70 |
| 10-9 | M/41 | B | 0 | 50 | 65 | 80 |
| 10-10 | M/43 | B | 0 | 50 | 75 | 75 |
| Average | | B | 0 | 50 | 65 | 72 |
| 08-11 | M/35 | C | 0 | 50 | 30 | 50 |
| 08-12 | M/31 | C | 0 | 30 | 30 | 50 |
| 08-13 | M/26 | C | 0 | 40 | 50 | 50 |
| 08-14 | F/17 | C | 0 | 40 | 40 | 60 |
| 08-15 | F/18 | C | 0 | 50 | 50 | 60 |
| Average | | C | 0 | 42 | 40 | 54 |
| 09-16 | F/29 | D | 0 | 30 | 25 | 60 |
| 09-17 | M/42 | D | 0 | 40 | 30 | 25 |
| 09-18 | M/51 | D | 0 | 40 | 25 | 30 |
| 09-19 | F/29 | D | 0 | 30 | 50 | 30 |
| 09-20 | F/23 | D | 0 | 40 | 40 | 40 |
| Average | | D | 0 | 36 | 34 | 37 |
| 09-21 | M/36 | E | 0 | 0 | 5 | 0 |
| 09-22 | M/39 | E | 0 | 0 | 0 | 0 |
| 09-23 | F/51 | E | 0 | 0 | 0 | 0 |
| 09-24 | M49 | E | 0 | 0 | 10 | 0 |
| 09-25 | F/38 | E | 0 | 0 | 0 | 0 |
| Average | 42.6 | | 0 | 0 | 3 | 0 |

Polygoni Cuspidati extract and licorice extract, then mix to obtain traditional Chinese medicine composition in Formula D.

### Embodiment 5

### Study of the Clinical Efficacy of Formula A, B, C, D Traditional Chinese Medicine Composition on Oral Ulcer Patients

Take Formula A, B, C, D traditional Chinese medicine composition from Embodiment 1-4 to dissolve into 30% (volume) glycerin's water solution to obtain liquid preparation, the contrast group (as E) only use 30% (volume) glycerin's water solution, respectively drip at the patient's oral ulcer place and take orally, the dosages are all 20 mg/kg (same dosage for Formula A, B, C, D), three times each day, continue for three consecutive days. Fill in the questionnaire based on patients' objective experience of pain alleviation, investigate the extent of pain alleviation, meanwhile inspect the wound surface of cured ulcer, the results are shown in Table 1, Formula A, B, C, D traditional Chinese medicine compositions all can cure oral ulcer and alleviate patients pain, but Formula with more varieties of crude drugs obviously have higher synergy effects, whereas the contrast group (E) has no effect.

### Embodiment 6

### The Anti-Tumor Effect of Traditional Chinese Medicine Composition in Formula A, B, C, D When Used in Conjunction with Chemotherapy Drugs

According to methods described in “Methodology in Pharmacological Research of Traditional Chinese Medicine” (People's Medical Publishing House), test the anti-tumor effects of Formula A, B, C, D traditional Chinese medicine composition prepared from Embodiment 1-4 along with chemotherapy drugs (5-fluorouracil) for human cancer xenograft tumor animal model. 5-fluorouracil (fu) is broad spectrum anti-tumor medicine, it is mainly used to treat digestive tract tumor, commonly seen adverse reactions include nausea, reduced appetite or vomit, abdomen discomfort or diarrhea. About the test method, use human colon cancer cell strain colon 38 to inoculate subcutaneously in 18-23 g mouse, 14 days after the inoculation, respectively ingest Formula A, B, C, D traditional Chinese medicine composition prepared from Embodiment 1-4 into the stomach of each group of animals, the dosage is 500 mg/kg; 0.5-1 hour after stomach ingestion, inject 5-fluorouracil into abdomen, the dosage is 30 mg/kg. Execute stomach ingestion and drug injection twice each week, totaling 4 weeks. The results after 4 weeks are listed in Table 2. According to the result, although the efficacy of traditional Chinese medicine composition in Formula A in suppressing tumor is less effective to that of 5-fluorouracil, when it is used in conjunction with 5-fluorouracil, the traditional Chinese medicine composition in Formula A, B, C, D can all reduce the side-effect of 5-fluorouracil intake (illustrated in death toll); also, the traditional Chinese medicine composition in Formula A, B, C can all enhance the effect in suppressing tumor, formula with more kinds of crude drugs obviously has higher synergetic effects for therapeutic effects.

TABLE 2
Tumor growth suppressing effects

| | | Average | Tumor | | |
|---|---|---|---|---|---|
| | | tumor | inhibition | Death | Supplied |
| Group | Drug | weight (g) | rate (%)* | number | number |
| CK | None (contrast | 1.16 ± 0.16 | — | 0 | 10 |
| | group) | | | | |
| CK1 | 30 mg/kg fu | 0.42 ± 0.11 | 64% | 5 | 10 |
| A | A/fu | 0.23 ± 0.13 | 80% | 0 | 10 |
| B | B/fu | 0.28 ± 0.12 | 76% | 0 | 10 |
| C | C/fu | 0.35 ± 0.15 | 70% | 1 | 10 |
| D | D/fu | 0.42 ± 0.13 | 64% | 1 | 10 |
| CK3 | A | 0.64 ± 0.11 | 45% | 0 | 10 |

*Tumor inhibition rate (%) = (CK group's average tumor weight − test group's average tumor weight)/contrast CK average tumor weight × 100%

1. Traditional Chinese medicine composition consisting of the extracts of no more than five crude drugs, in which the crude drugs include licorice and also include Radix Scutellariae or Rhizoma Polygoni Cuspidati.

2. The composition as described in claim 1, wherein the crude drugs include Radix Scutellariae, Rhizoma Polygoni Cuspidati, licorice, Radix puerariae and Semen Celosiae.

3. The composition as described in Claim 1, wherein the weight ratio of crude drugs is:
licorice 0.5-2.5 portions, Radix Scutellariae 1-5 portions, Rhizoma Polygoni Cuspidati 0.5-5 portions, Radix puerariae 0.2-2.5 portions, and/or Semen Celosiae 0.3-2.5 portions.

4. The composition as described in claim 3, wherein the weight ratio of crude drugs is: Radix Scutellariae 2.5 portions, Rhizoma Polygoni Cuspidati 2.5 portions, licorice 1.25 portions, Radix puerariae 1.25 portions, and Semen Celosiae 1.25 portions.

5. The composition as described in Claim 1, wherein, Radix Scutellariae extract has a baicalin content which exceeds 10% (weight); Rhizoma Polygoni Cuspidati extract has a polydatin content which exceeds 1% (weight); and licorice extract has a licoflavone content which exceeds 4% (weight).

6. Traditional Chinese medicine preparation, which includes the composition described in claim 1 and a pharmaceutically acceptable adjuvant.

7. The preparation described in claim 6, wherein the preparation is an oral intake preparation in the form of a spray, tablet, capsule or oral intake solution.

8. The usage of composition described in claim 1 in a prepared drug, wherein the drug is used to treat oral ulcer, or for treating tumor.

9. A method to treat oral ulcer or tumor, which includes administering an effective dose of composition described in claim 1 for treating patients.

10. A method to prepare the compositions described in claim 1, comprising:
(1) extract Radix Scutellariae crude drug to obtain Radix Scutellariae extract through an acid precipitation method;
(2) extract Rhizoma Polygoni Cuspidati crude drug to obtain Rhizoma Polygoni Cuspidati extract through an ethanol extraction method;
(3) extract licorice crude drug to obtain licorice extract through an ethanol extraction method;
(4) extract Radix puerariae crude drug to obtain Radix puerariae extract through an ethanol extraction method or water extraction method;
(5) extract Semen Celosiae crude drug to obtain Semen Celosiae extract through an ethanol extraction method or water extraction method; and
(6) mix extracts obtained from Step (3) and (1) and/or (2) and/or (4) and/or (5) and/or (6).

11. The composition of claim 1, wherein the crude drugs include Radix Scutellariae, Rhizoma Polygoni Cuspidati and licorice.

12. The composition as described in claim 1, wherein Radix Scutellariae extract has a baicalin content which exceeds 50% (weight); Rhizoma Polygoni Cuspidati extract has a polydatin content which exceeds 2% (weight); and licorice extract has a licoflavone content which exceeds 4% (weight).

13. A traditional Chinese medicine composition, consisting of at least two extracts of crude drugs selected from the group consisting of licorice, Radix Scutellariae, Rhizoma Polygoni Cuspidati, Radix puerariae and Semen Celosiae, and wherein the at least two extracts include extracts of at least licorice and Radix Scutellariae.
